(19) United States
(12) Patent Application Publication
(10) Pub. No.: US 2024/0254528 A1
(43) Pub. Date: Aug. 1, 2024

(54) AQUEOUS BIOLOGICAL SYSTEMS WITH REDUCED PHOSPHATE LEVELS AND METHODS OF REDUCING PHOSPHATE LEVELS

(71) Applicant: CORN PRODUCTS DEVELOPMENT, INC., WESTCHESTER, IL (US)

(72) Inventor: Dmitry KAZACHKIN, Bridgewater, NJ (US)

(21) Appl. No.: 18/561,054

(22) PCT Filed: May 25, 2022
(86) PCT No.: PCT/US2022/030803

§ 371 Date: Nov. 15, 2023

Related U.S. Application Data
(60) Provisional application No. 63/194,499, filed on May 28, 2021.

(51) Int. Cl. C12P 19/02; C07H 1/08; C12N 1/06

(52) U.S. Cl. CPC C12P 19/02; C07H 1/08; C12N 1/06

(57) ABSTRACT

The technology disclosed in this specification pertains to methods of reducing phosphate levels in aqueous preparations containing phosphate with the admixture of first and second compounds containing cations which can bind phosphates. In any embodiment, the phosphate level is reduced to less than about 80 wt. %. The technology also relates to methods of collecting and sequestering phosphate in aqueous preparations. The technology also relates to the collection or removal of phosphate while minimizing the loss of desired target molecules, such as saccharides. In any embodiment, the aqueous preparation is maintained within certain pH ranges during the admixture of the first and second compounds, to maintain the stability of certain target molecules, such as saccharides, during the reduction of the phosphate. Also disclosed is an aqueous preparation having precipitated phosphate or reduced phosphate levels, particularly aqueous preparations also maintaining levels of desired saccharides.

The present invention relates to methods of reducing phosphate levels in aqueous systems. It also relates to methods of reducing phosphates while retaining desired target molecules in the aqueous system. This invention also relates to aqueous systems more specifically to biological aqueous systems having reduced phosphate levels and little or no loss of certain target molecules.

Biological aqueous systems can provide a mixture of components suitable for synthesizing or modifying target molecules wherein phosphate is present. Phosphate occurs in biological and chemical aqueous systems in a variety of ways including, for example, as an endogenous component, as a waste product generated by certain enzymatic and chemical reactions, and as a required cofactor added to facilitate certain enzymatic and chemical reactions. While phosphate may be a requirement for certain reactions, the absence of phosphate (or the presence of phosphate below a threshold level) may be required for subsequent, downstream reactions. Accordingly, there is often a need to reduce phosphate concentrations in biological systems so that the biological system remains competent to facilitate any of a variety of downstream biological or chemical reactions.

It may also be desired to reduce phosphate levels to facilitate the recovery of certain target molecules also present in the aqueous biological and chemical systems. Many target molecules are vulnerable to degradation by the harsh effects of certain agents commonly used to that remove, or chelate, phosphate. There is a further need to provide for phosphate reduction in aqueous systems that facilitates the production or recovery of certain target molecules that co-reside with the phosphate before its removal from the system, while simultaneously maintaining the aqueous system at a pH conducive to the preservation or retention of those target molecules, particularly saccharides such as allulose or tagatose. Further, it may be desirable to achieve such effects using readily available and inexpensive compounds, to provide a cost-effective and convenient method for phosphate reductions.

This specification describes methods of treating phosphate-containing biological systems, using a combination of compounds that each contain a polyvalent cation. As disclosed herein, the degree of phosphate reduction can be improved by the use of multiple compounds that contain polyvalent cations, compared to the addition of a single polyvalent cation-containing compound. However, a non-linear relationship was identified between amounts of multiple polyvalent cations required for contemporaneous phosphate reduction and the retention of certain target molecules, such as allulose, in a biological aqueous system.

The specification also describes aqueous systems having reduced phosphate levels. Also disclosed are aqueous systems having reduced levels of phosphate levels reduced and substantially unreduced levels of a desired target molecule, such as allulose, as well as aqueous systems having phosphate segregated from the desired target molecules.

### BRIEF DESCRIPTION OF THE DRAWINGS

FIG. 1 shows the phosphate concentrations measured in samples after treatment with one or more compounds containing polyvalent cations. Treated samples were subjected to the addition of CaCl₂), (samples 7 and 10), the addition of Ca(OH)₂, (samples 4, 11, 13), or a combination of both compounds (samples 1, 3, 5-6, 8-9, 12, and 14), compared to untreated control samples (samples 0a, 0b, 2, and 15). The phosphate concentrations of the treated samples were measured in duplicate.

FIG. 2 shows the amounts of monosaccharides (allulose, fructose, dextrose) present in samples of FIG. 1 after single- and multi-polyvalent compound treatments for reducing phosphate levels.

### DETAILED DESCRIPTION OF EMBODIMENTS OF THE INVENTION

The present technology is also not to be limited in terms of the aspects described herein, which are intended as illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing aspects only and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

The following definitions and comments are useful for interpreting the various embodiments of the technologies disclosed in this specification.

Reference in this specification to “polyvalent compound” means a compound containing one or more polyvalent components, the compound dissolving in water or other solvent to release the polyvalent components. The term encompasses compounds containing anionic components, cationic compounds, and mixtures thereof. The term encompasses compounds containing polyvalent anionic components, polyvalent cationic compounds, and mixtures thereof. The polyvalent compound may have any positive or negative charge or no net charge at all. In any embodiment, the polyvalent compound provides for the release of at least one ionic component.

Reference in this specification to “polyvalent cation” means an ion having a net positive charge greater than or equal to two. Reference in this specification to a “polyvalent anion” means an ion having a net negative charge greater than or equal to two. In any embodiment, a polyvalent compound can contain one or more polyvalent cations, one or more polyvalent anions, or mixtures thereof.

Reference in this specification to “monovalent compound” means a compound containing one or more monovalent components, the compound dissolving in water or other solvent to release the monovalent components. The term encompasses compounds containing anionic components, cationic compounds, and mixtures thereof. The monovalent compound may have any positive or negative charge or no net charge at all. In any embodiment, the monovalent compound provides for the release of at least one ionic component.

Reference in this specification to “monovalent cation” means an ion having a net positive charge equal to one. Reference in this specification to a “monovalent anion” means an ion having a net negative charge equal to one. In any embodiment, a monovalent compound can contain one or more monovalent cations, one or more monovalent anions, or mixtures thereof.

Reference in this specification to the “phosphate assay” means an assay based on the malachite green colorimetric method for measuring phosphate release, such as reported by Baykov et al. as reported in “A Malachite Green Procedure for Orthophosphate Determination and its use in Alkaline Phosphatase-Based Enzyme Immunoassay” 172 Analytical Biochemistry 266-270 (1988). The phosphate levels present in various solutions were measured with a Phosphate Assay Kit according to the instructions of the manufacturer (Sigma-Aldrich). To quantify phosphate concentrations of samples, aliquots were taken at different times and mixed with malachite green buffers as described by the manufacturer. After 30 min of incubation, the O.D. at 620 nm was determined using a SpectraMax iD3 spectrophotometer (Molecular Devices). A standard curve with free phosphate was produced according to the instructions of the manufacturer.

Reference in this specification to the “saccharide assay” means an in vitro assay useful for estimating the amount or concentration of saccharide in an aqueous system. For example, an “allulose assay” means an assay for estimating the amount or concentration of allulose in the aqueous system.

Reference in this specification to “target molecules” are large molecules composed of covalently connected atoms. In biological systems, target molecules include, but are not limited to, carbohydrates, lipids, proteins, saccharides, and nucleic acids. Though often considered biological in nature, such target molecules are found in other systems, such as chemical systems.

Reference in this specification to “saccharides,” means target molecules having the general formula of C(H₂O)n, where n is an integer. Saccharides include “monosaccharides,” where the saccharides are carbohydrates having between 3-7 carbon atoms. Saccharides include “polysaccharides,” polymers of monosaccharide sugars covalently linked together, saccharides having a greater number of carbon atoms than monosaccharides.

Reference in this specification to “biological aqueous system,” means an aqueous solution containing components typically found in a biological system (e.g., cell), such as, but not limited to phosphate, chemicals, and target molecules. Although embodiments of the disclosed invention encompass aqueous systems considered primarily biological in nature, such embodiments are equally applicable to other systems containing such component, including but not limited to chemical, biochemical, physical, and non-biological, and mixtures thereof.

Reference in this specification to a “cell-free system” means an in vitro tool used to study biological reactions occurring within cells, or associated with cells. An example of such biological process includes the synthesis of a biomolecule or chemical compound without using intact, living cells. Instead, the cells are lysed and portions of the cell lysate, often containing competent enzymes, are used to make a desired biological or chemical product. As such, the term is understood to mean a system made from less-than-complete cells, which reduces the complex interactions typically found in a whole cell, but providing a simplified analog of complete and intact cells.

Reference in this specification to a “precipitate,” means the conversion of a chemical substance in a liquid solution or aqueous system into a solid, typically done by converting the chemical substance into an insoluble form. Precipitation can also occur when soluble substances interact to form insoluble complexes of those otherwise-soluble substances.

Use of “about” to modify a number is meant to include the number recited plus or minus 10%. Where legally permissible recitation of a value in a claim means about the value. Use of “about” in a claim or in the specification is not intended to limit the full scope of covered equivalents.

Recitation of the indefinite article “a” or the definite article “the” is meant to mean one or more unless the context clearly dictates otherwise.

In any embodiment, first and second polyvalent compounds as disclosed in this specification, each provides a content of at least about 0.5 wt. % to about 1.5 wt. % of the aqueous system and together provides a reduction or precipitation of at least about 80% in the phosphate concentration in aqueous system.

The technology disclosed in this specification pertains to methods of making a reduced-phosphate aqueous system by a) obtaining an aqueous preparation, the aqueous preparation containing phosphate; b) combining the aqueous preparation with first and second polyvalent compounds to provide a treated preparation, wherein each polyvalent compound contains a polyvalent cation; c) mixing the treated preparation at sufficient temperature and for sufficient time for the phosphate and the polyvalent cations to combine as a precipitate or complex; and d) optionally, removing the precipitate or complex from the treated preparation to provide the reduced-phosphate preparation.

In any embodiment, the method is carried out at a pH that is optimal to facilitate precipitation of phosphates, and to preserve or maintain the levels of desired target molecules in the aqueous preparation. In any embodiment, the method is carried out at a pH that is optimal to maximize precipitation of phosphates, yet also prevent undesired biological or chemical conversions, such as but not limited to the irreversible conversion of allulose to other saccharides or other molecules. In any embodiment the aqueous preparation, as described in this specification, has a pH between about 2 and about 12, between about 4 and about 10, between about 6 and about 10, between about 6 and about 9, between about 6 and about 8, between about 6 and about 7, between about 7 and about 10, between about 8 and about 10, between about 9 and about 10, or about 12, or about 10, or about 9, or about 8. In any embodiment the aqueous preparation has a pH between about 4 and about 10, or between about 5.5 and about 8.5. In any embodiment the aqueous preparation has a pH about 8.

In any embodiment, the first and second polyvalent compounds, as described in this specification, each contain a polyvalent cation. In any embodiment, each polyvalent compound comprises a salt, the salt comprising a polyvalent cation complexed with one or more mono-, di-, or polyvalent anions. In any embodiment, each polyvalent cation comprises a net positive charge of 1+, 2+, 3+, 4+, or greater; or of 2+, 3+, 4+, or greater. In any embodiment, both first and second polyvalent cations comprise a net positive charge of 1+, 2+, 3+, 4+, or greater; of 2+, 3+, 4+, or greater. In any embodiment, each polyvalent cation has the same charge.

In any embodiment, each polyvalent cation comprises a net positive charge greater than or equal to 2. In any embodiment, each polyvalent cation comprises a net positive charge about equal to 2. In preferred embodiments, the first and second polyvalent cations each comprises a net positive charge greater than or equal to 2. In any embodiment, the first and second polyvalent cations both have a net positive charge greater than or equal to 2.

In any embodiment, each polyvalent cation is a metal ion, transition metal ion, and/or alkaline earth metal ion, or a mixture thereof. In any embodiment, the polyvalent cation is calcium, magnesium, barium, zinc, iron, iron, copper, aluminum, lead, silver, titanium or a mixture thereof. In any embodiment, the polyvalent cation is calcium. In any embodiment, the polyvalent cation is divalent. The divalent cation may be selected from calcium, zinc, magnesium, and titanium. In certain embodiments, the divalent cation is calcium. In certain embodiments, the polyvalent cation is trivalent. The trivalent cation may be selected from aluminum, cobalt, and iron.

In any embodiment, the first polyvalent compound comprises a first polyvalent cation and the second polyvalent compound comprises a second polyvalent cation. In any embodiment, the first and second polyvalent cations have the same or different net positive charge. In any embodiment, the first and second polyvalent cations comprise the same or different polyvalent cations or mixtures thereof. In preferred embodiments, the first and second polyvalent compounds comprise the same polyvalent cation.

In any embodiment, each polyvalent compound is or comprises an inorganic compound. In preferred embodiments, each polyvalent compound is soluble in water. In any embodiment, each polyvalent compound has moderate solubility in water, wherein about 10 to about 1,000 mg of the compound is soluble in water or is present in an amount of about 10 to about 1,000 parts per million (ppm). In any embodiment, each polyvalent compound has high solubility in water, wherein greater than about 1,000 mg of the compound is soluble in water or is present in an amount of greater than about 1,000 parts per million (ppm).

In any embodiment, each polyvalent compound is or comprises a polyvalent compound selected from aluminum chloride, lanthanum chloride, calcium bromide, calcium chloride, calcium hydroxide, calcium oxide, calcium nitrate, ferric chloride, iron hydroxide, cesium nitrate, cesium chloride, cesium bromide, magnesium chloride, magnesium hydroxide, magnesium oxide, hydrogen chloride, sulfuric acid, ammonium hydroxide, sodium hydroxide, potassium hydroxide, zinc chloride, zinc bromide, and mixtures thereof. In any embodiment each of the first and second compounds is selected from aluminum chloride, calcium bromide, calcium chloride, calcium hydroxide, calcium oxide, calcium nitrate, ferric chloride, iron hydroxide, magnesium chloride, magnesium hydroxide, magnesium oxide, magnesium bromide, hydrogen chloride, sulfuric acid, ammonium hydroxide, sodium hydroxide, potassium hydroxide, zinc chloride, zinc bromide, and mixtures thereof. In any embodiment, the first and second compounds are selected from calcium chloride, calcium hydroxide, and calcium oxide. In any embodiment, the first and second compounds are selected from calcium chloride and calcium hydroxide.

In aqueous systems, phosphate precipitation occurs most efficiently within certain pH ranges. For any given system, the method is carried out at a pH that is optimal to facilitate precipitation of phosphates, while also preserving or maintaining the levels of desired target molecules in the aqueous preparation, such as certain saccharides. For the given system, the pH is targeted to maximize precipitation of phosphates and to minimize degradation of target molecules, such as allulose. For the given system, the pH is targeted to maximize precipitation of phosphates and to minimize conversion of target molecules into other molecules. In any embodiment, the aqueous preparation is maintained at a pH at or above about 2, 3, 4, 5, 6, or 7.

In aqueous systems, certain biological and chemical reactions occur only upon reaching certain pHs. For example, to prevent undesired chemical or biological reactions, the aqueous system is maintained within a particular pH or pH range while the polyvalent compounds are added to the aqueous system. In any embodiment, the aqueous preparation, as described in this specification, is maintained at a pH between about 2 and about 12, between about 4 and about 10, between about 6 and about 10, between about 6 and about 9, between about 6 and about 8, between about 6 and about 7, between about 7 and about 10, between about 8 and about 10, between about 9 and about 10, or about 12, or about 10, or about 9, or about 8, during the addition of the polyvalent compounds. In any embodiment the aqueous preparation is maintained at a pH between about 4 and about 10, or between about 5.5 and about 8.5 during the addition of the polyvalent compounds. In any embodiment the aqueous preparation has a pH of about 8 during the addition of the polyvalent compounds.

In any embodiment, the first and second polyvalent or monovalent compounds are added in proportions to maximize precipitation of the phosphate in the aqueous system while at the same time preventing side reactions such as degradation of sugars of interest (allulose). In any embodiment, the first polyvalent compound is added to the aqueous preparation at an amount so as to be between about 0.001 wt. % to about 10.0 wt. % of the treated preparation, or about 0.01 wt. % to about 10.0 wt. %, about 0.1 wt. % to about 5.0 wt. %, about 0.5 wt. % to about 2.0 wt. %, or about 0.50 wt. % to about 1.5 wt. %, or about 0.50 wt. % to about 1.0 wt. % of the treated preparation. In any embodiment, the first polyvalent compound is added to the aqueous preparation at an amount so as to be between about 0.50 wt. % to about 1.5 wt. % of the treated preparation, or between about 0.50 wt. % to about 1.0 wt. %, or between about 0.75 wt. % to about 1.0 wt. %. In any embodiment, the first polyvalent compound is added to the aqueous preparation at an amount so as to be between about 0.75 wt. % to about 1.0 wt. %.

In any embodiment, the second polyvalent compound is added to the aqueous preparation at an amount between about 0.001 wt. % to about 10.0 wt. % of the treated preparation, or about 0.01 wt. % to about 10.0 wt. %, about 0.1 wt. % to about 5.0 wt. %, about 0.5 wt. % to about 2.0 wt. %, or about 0.50 wt. % to about 1.5 wt. %, or about 0.50 wt. % to about 1.0 wt. % of the treated preparation. In any embodiment, the second polyvalent compound is added to the aqueous preparation at an amount so as to be between about 0.25 wt. % to about 1.5 wt. % of the treated preparation, or between about 0.5 wt. % to about 1.0 wt. % of the treated preparation. In any embodiment, the second polyvalent compound is added to the aqueous preparation at an amount between about 0.5 wt. % to about 1.5 wt. % of the treated preparation.

In any embodiment, the combined polyvalent compounds provide an amount between about 0.001 wt. % to about 10.0 wt. % of the treated preparation, or about 0.01 wt. % to about 10.0 wt. %, about 0.1 wt. % to about 5.0 wt. %, about 0.5 wt. % to about 2.0 wt. %, or about 0.50 wt. % to about 1.5 wt. %, or about 0.50 wt. % to about 1.0 wt. % of the treated preparation. In any embodiment, the combined polyvalent compounds provide an amount between about 1.0 wt. % to about 10.0 wt. % of the treated preparation, or between about 1 wt. % to about 5.0 wt. %, or between about 1 wt. % to about 2.0 wt. %.

In any embodiment, the first and second polyvalent compounds, as described in this specification are added to the aqueous preparation in a ratio between about 0.5:1 to about 10:1, between about 0.5:1 to about 9:1, between about 0.5:1 to about 8:1, between about 0.5:1 to about 7:1, between about 0.5:1 to about 6:1, between about 0.5:1 to about 5:1, between about 0.5:1 to about 5:1, between about 0.5:1 to about 4:1, between about 0.5:1 to about 3:1, between about 0.5:1 to about 2:1, between about 0.5:1 to about 1:1, or about 1:1, or about 1:1.2, or about 1:1.4, or about 1:1.6, or about 1:1.8, or about 1:1.2 relative to each other. In any embodiment, the first and second polyvalent compounds are added in a ratio between about 0.75:1 to about 1.5:1. In any embodiment, the first and second polyvalent compounds are added in a ratio between about 1.5:1.

The technology disclosed in this specification also pertains to methods of making a reduced-phosphate aqueous system by a) obtaining the aqueous preparation comprising phosphate and a target molecule; b) combining the aqueous preparation with a first compound and a second compound, to provide a treated preparation, wherein at least one of the compounds comprises a polyvalent cation; c) mixing the treated preparation at sufficient temperature and for sufficient time for the phosphate and the compounds to form a precipitate, to provide a reduced-phosphate preparation; and d) optionally, removing the precipitate from the reduced-phosphate preparation; wherein the reduced-phosphate preparation retains a portion of the target molecule.

In any embodiment, both of the first and second compounds comprise a polyvalent cation or polyvalent anion. In any embodiment, both of the first and second compounds comprise a polyvalent cation. In any embodiment, at least one of the first and second compounds comprise a polyvalent cation. In any embodiment, at least one of the first and second compounds comprise a monovalent cation.

In any embodiment, the second compound comprises or is a monovalent compound. In any embodiment, each monovalent compound is or comprises a monovalent compound selected from sodium hydroxide, sodium carbonate, potassium hydroxide, ammonium hydroxide, or any other organic or inorganic acid or salt capable increasing pH (reduced proton activity). In any embodiment, each monovalent compound is or comprises a monovalent compound selected from hydrochloric acid, chloric acid, perchloric acid, nitric acid, acetic acid, or any organic or inorganic acid or salt capable to reduce pH (increase proton activity). In any further embodiment, the monovalent compound is selected from sodium hydroxide and hydrochloric acid calcium hydroxide.

In any embodiment, a monovalent cation is selected from hydrogen, lithium, sodium, potassium, rubidium, cesium, francium. In any embodiment, a monovalent anion is selected from fluoride, chloride, bromide, nitrite, acetate, formate, iodine, or hydroxide.

In any embodiment, the second compound is a second polyvalent compound.

In any embodiment, the aqueous preparation, as described in this specification, contains soluble phosphate. In any embodiment, the aqueous preparation comprises soluble phosphate at a concentration between about 1 and about 1000 mM, between about 1 and about 500 mM, between about 1 and about 250 mM, between about 1 and about 100 mM, between about 10 and about 1000 mM, between about 10 and about 500 mM, between about 10 and about 250 mM, between about 10 and about 100 mM, or between about 10 and about 50 mM. In any embodiment, the aqueous preparation comprises soluble phosphate at a concentration between about 1 and about 100 mM, between about 10 and about 50 mM, or between about 10 and about 30 mM. In any embodiment, the aqueous preparation comprises soluble phosphate at a concentration between about 1 and about 100 mM, or between about 10 and about 30 mM.

In any embodiment, the aqueous preparation, as described in this specification, contains soluble phosphate. In any embodiment, the aqueous preparation comprises soluble phosphate in an amount between about 0.0001% and about 10% of the weight of the aqueous solution, or between about 0.0001% and about 1%, between about 0.0001% and about 0.1%, between about 0.0001% and about 0.01%, between about 0.0001% and about 0.001% of the weight of the aqueous solution.

In any embodiment, the treated preparation, as described in this specification, has a pH between about 2 and about 12, between about 4 and about 10, between about 6 and about 10, between about 8 and about 10, between about 9 and about 10, or below about 12, or below about 10, or below about 9, or below about 8. In preferred embodiments, the pH is a physiological pH, that is a pH normally found in a prokaryotic body, tissue, or cell. In more preferred embodiments, the pH is a physiological pH between about 7 and about 9, or between about 7 and about 8, or between about 7.2 to about 7.6, or about 7.5. In any embodiment, the treated preparation has a pH between about 6 and about 12, between about 6 and about 10, or between about 7 and about 10. In any embodiment, the treated preparation has a pH between about 6 and about 10. In any embodiment, the treated preparation has a pH about 8.

In any embodiment, a treated preparation, as described above, is an aqueous composition having a pH that is adjusted to a pH between about 4 and about 10, between about 6 and about 10, between about 8 and about 10, between about 9 and about 10, or below about 12. In any embodiment, the treated preparation has a pH between about 6 and about 12, between about 6 and about 10, or between about 7 and about 10. In any embodiment, the treated preparation has a pH between about 6 and about 10. In any embodiment, the treated preparation has a pH about 8. In any embodiment the pH of the treated preparation is obtained by using any appropriate grade acid, including but not limited to hydrochloric acid, or any appropriate grade base, including but not limited to sodium hydroxide.

In any embodiment, a treated preparation is achieved by combining an aqueous preparations with one or more polyvalent compounds. In any embodiment, the treated preparation maintains a pH similar to that of the aqueous preparation before the addition of the polyvalent compounds. In any embodiment, the treated preparation maintains such similar pH during the admixture of the polyvalent compounds into the aqueous solution. In any embodiment, the treated preparation maintains a pH that is within about 1 to about 6 pH units of the pH of the aqueous solution before the addition of the polyvalent compounds, or about 1 to about 5 pH units, or about 1 to about 4 pH units, about 1 to about 3 pH units, about 1 to about 2 pH units.

In some embodiment, the treated preparation retains a certain pH during the admixture of the aqueous preparations with one or more polyvalent compounds. In any embodiment, the treated preparation maintains a pH between about 2 and about 12, between about 4 and about 10, between about 6 and about 10, between about 6 and about 9, between about 6 and about 8, between about 6 and about 7, between about 7 and about 10, between about 8 and about 10, between about 9 and about 10, or about 12, or about 10, or about 9, or about 8. In any embodiment, the treated preparation maintains a pH below about 12, below about 10, below about 8, below about 6, below about 4, or below about 3. In any embodiment, the treated preparation maintains a pH above about 2, above about 4, above about 6, above about 8, above about 10, or above about 12. In any embodiment, the treated preparation maintains a pH between about 6 and about 12, between about 6 and about 10, or between about 7 and about 10. In any embodiment, the treated preparation maintains a pH between about 8.

In any embodiment, the pH of the treated preparation is maintained without the addition of additional agents for adjusting pH, such as concentrated or dilute acids and bases. In any embodiment, such pH adjusting agent includes acidic agents such as but not limited to hydrochloric acid and sulfuric acid. In any embodiment, such pH adjusting agent includes basic agents such as but not limited to sodium hydroxide, sodium carbonate, ammonium hydroxide, calcium hydroxide, and magnesium hydroxide.

In any embodiment, a treated preparation, as described above, the treated preparation is mixed at sufficient temperature and for sufficient time for the phosphate and the polyvalent compounds to combine as a precipitate. In any embodiment, the mixing step is performed at a temperature between about 10° C. and about 200° C., between about 10° C. and about 150° C., between about 10° C. and about 120° C., between about 10° C. and about 100° C., between about 20° C. and about 100° C., between about 20° C. and about 80° C., between about 40° C. and about 80° C., between about 60° C. and about 80° C., between about 60° C. and about 70° C., or between about 10° C. and about 100° C., or about 60° C., or about 65° C., or about 70° C., or about 75° C.

In any embodiment, the mixing step is performed at a temperature between about 0° C. and about 10° C., or between about 20° C. and about 90° C., or between about 100° C. and about 150° C. In any embodiment, the mixing step is performed at a temperature between about 0° C. and about 10° C. In any embodiment, the mixing step is performed at a temperature between about 20° C. and about 90° C. In any embodiment, the mixing step is performed at a temperature between about 100° C. and about 150° C.

Where pressure or compression is applied to the aqueous preparation, the mixing step can be performed at higher temperature. In any embodiment, the mixing step is performed at a temperature between about 10° C. and about 200° C., between about 10° C. and about 150° C., between about 10° C. and about 120° C., between about 20° C. and about 100° C., between about 40° C. and about 80° C., between about 60° C. and about 80° C., between about 60° C. and about 70° C., or between about 10° C. and about 100° C., or about 60° C., or about 65° C., or about 70° C., or about 75° C.

In any embodiment, the mixing step (step b) is performed for a time between about 1 minute to about 60 minutes, between 5 minutes to about 60 minutes, between about 10 minutes to about 60 minutes, between about 15 minutes to about 60 minutes, between about 10 minutes to about 45 minutes, between about 10 minutes to about 30 minutes, or about 5 minutes, about 10 minutes, about 15 minutes, about 20 minutes, about 30 minutes, or about 60 minutes.

In any embodiment, after the treated preparation is mixed at sufficient temperature and for sufficient time for the phosphate and the polyvalent compounds to combine as a precipitate, the precipitate that forms comprises from about 1 wt. % to about 100 wt. % of the phosphate in the treated solution, or from about 1 wt. % to about 90 wt. %, or from about 1 wt. % to about 80 wt. %, or from about 1 wt. % to about 70 wt. %, or from about 1 wt. % to about 60 wt. %, or from about 10 wt. % to about 100 wt. %, from about 10 wt. % to about 90 wt. %, or from about 10 wt. % to about 80 wt. %, or from about 10 wt. % to about 70 wt. %, or from about 10 wt. % to about 50 wt. %, or from about 50 wt. % to about 100 wt. %, or from about 50 wt. % to about 90 wt. %, or from about 50 wt. % to about 80 wt. % of the phosphate in the treated solution.

In any embodiment, the precipitate further comprises one or more target molecules. In any embodiment, the target molecule includes a cellular component such as but not limited to nucleic acids, carbohydrates, lipids, saccharides, proteins, and proteoglycans, or combinations thereof. In any embodiment, the target molecule is a nucleic acid, such as DNA, RNA, etc. In any embodiment, such target molecules include fibers such as fibers from any resistant starches, and soluble fibers such as polydextrose or short chain fructooligosaccharides. In any embodiment, the target molecule is a saccharide such as allulose, allose, tagatose, glucose, fructose, sorbitol, ribulose, ribose, arabinose, lyxose, xylose, ribulose, xylulose, allose, altrose, galactose, gulose, idose, mannose, talose, dextrose, and sorbose. In any embodiment, the target molecule is a saccharide such as fructose, dextrose, allulose, or tagatose. In any embodiment, the target molecule is a saccharide such as allulose or tagatose.

In any embodiment, the precipitate, as described in this specification, can be collected by any suitable recovery process. In any embodiment, precipitate (or solid portion of the treated preparation) is removed, recovered, or separated from the aqueous portion of the treated preparation (e.g., supernatant). In any embodiment, the phosphate-reduced aqueous portion can be separated or recovered from the phosphate-rich precipitate by a process selected from, but not limited to, the following: filtration, centrifugation, precipitation, and combinations thereof. In any embodiment where the precipitate is removed by centrifugation, that process can be performed under the following conditions: between about 100×g to about 100,000×g, between about 500×g to about 50,000×g, between about 1000×g to about 20,000×g, or between about 1000×g to about 10000×g, or about 500×g, or about 1000×g, or about 2000×g, or about 2500×g, or about 5000×g, for a range of average residence time between about 0.1 min to about 1 min, about 1 minute and about 60 minutes, about 5 minutes and about 30 minutes, about 5 minutes and about 15 minutes, about 1 minute, about 5 minutes, about 10 minutes, about 15 minutes, about 30 minutes, or about 60 minutes.

In any embodiment, the reduced-phosphate preparation has a reduction of the phosphate present compared to the aqueous preparation, the percentage of phosphate in the reduced-phosphate preparation being reduced in an amount between about 1% to about 100% compared to the aqueous preparation, between about 10% to about 100%, between about 50% to about 100%, between about 75% to about 100%, between about 90% to about 100%, between about 50% to about 100%, between about 50% to about 90%, or between about 50% to about 75%, compared to the aqueous preparation.

In any embodiment, the reduced-phosphate preparation retains a percentage of the phosphate from the aqueous preparation at between about 1% to about 20%, between about 1% to about 10%, between about 1% to about 5%, or between about 5% to about 10%. In any embodiment, the reduced-phosphate preparation retains between about 1% to about 20% of the phosphate present in the aqueous preparation, between about 1% to about 10%, between about 1% to about 5%, or between about 5% to about 10%. In any embodiment, the reduced-phosphate preparation retains between about 1% to about 20% of the phosphate present in the aqueous preparation, or between about 1% to about 10% of the phosphate present in the aqueous preparation. In any embodiment, the reduced-phosphate preparation retains a percentage of phosphate in the reduced-phosphate preparation at less than 10%, less than 5%, or less than 1%.

In any embodiment, the reduced-phosphate preparation has a reduction of the phosphate present in the aqueous preparation, the percentage of phosphate in the aqueous preparation being reduced in an amount between about 1% to about 100%, between about 10% to about 100%, between about 50% to about 100%, between about 75% to about 100%, between about 90% to about 100%, between about 50% to about 100%, between about 50% to about 90%, or between about 50% to about 75%, compared to the aqueous preparation.

In any embodiment, the percentage of phosphate in the precipitate is between about 1% to about 100% of the aqueous preparation, between about 10% to about 100%, between about 50% to about 100%, between about 75% to about 100%, between about 90% to about 100%, between about 50% to about 100%, between about 50% to about 90%, or between about 50% to about 75% of the aqueous preparation.

In any embodiment, the precipitate contains a percentage of the phosphate present in the aqueous preparation, the percentage of phosphate in the reduced-phosphate preparation being between about 1% to about 100%, between about 10% to about 100%, between about 50% to about 100%, between about 75% to about 100%, between about 90% to about 100%, between about 50% to about 100%, between about 50% to about 90%, or between about 50% to about 75%, compared to the aqueous preparation. In any embodiment, the precipitate contains phosphate at a percentage of between about 70% to about 100% of the phosphate present in the aqueous preparation, or between about 80% to about 100%, or between about 90% to about 100%. In any embodiment, the precipitate contains phosphate at a percentage of greater than about 90% of the phosphate present in the aqueous preparation.

In any embodiment, the precipitate is collected or concentrated. In any embodiment, the collected or concentrated precipitate is segregated or removed from the aqueous preparation. In any embodiment, the collected or concentrated precipitate remains in the aqueous preparation.

In any embodiment, it may be desired to collect or concentrate the precipitate without necessarily removing the precipitate from the treated aqueous system. In such embodiments, the precipitate is collected or concentrated without being removed or separated from the treated preparation. In any embodiment, it may be desired to create the precipitate to segregate the phosphate from other components in the aqueous system precipitate, without further need to segregate the precipitate from those components. In any such embodiment, the precipitate is formed without being collected or concentrated.

In any embodiment, the reduced-phosphate preparation retains reduced level or concentration of phosphate present in the aqueous preparation, the concentration of phosphate in the reduced-phosphate preparation being between about 0 mM to about 100 mM, or about 1 mM to about 100 mM, or about 1 mM to about 50 mM, or about 1 mM to about 30 mM, or about 1 mM to about 20 mM, or about 1 mM to about 15 mM, or about 1 mM to about 10 mM, or about 1 mM to about 5 mM, or less than about 25 mM, or less than about 20 mM, or less than about 15 mM, or less than about 10 mM or less than about 5 mM. In any embodiment, the reduced-phosphate preparation retains reduced level or concentration of phosphate present in the aqueous preparation, the concentration of phosphate in the reduced-phosphate preparation being between about 1 mM to about 10 mM, or about 1 mM to about 5 mM.

In any embodiment, the aqueous preparation can contain one or more target molecules. In any embodiment relating to cell-based, cell-free, or biological system, the target molecules may include cellular components such as but not limited to nucleic acids, carbohydrates, lipids, saccharides, proteins, and proteoglycans, or combinations thereof. In any embodiment, the target molecule may include or be a saccharide.

In any embodiment, the aqueous preparation comprises one or more target molecules. In any embodiment, the target molecule is a nucleic acid, such as DNA, RNA, etc. In any embodiment, such target molecules may include fibers such as fibers from any resistant starches, and soluble fibers such as polydextrose or short chain fructooligosaccharides. Some aspects of the present disclosure provide methods for obtaining a saccharide (such as allulose, allose, tagatose, glucose, fructose, sorbitol, ribulose, ribose, and/or arabinose) from a lysate obtained from one or more cell populations expressing at least one thermostable enzyme of a saccharide production or sugar conversion pathway. Some methods of the present disclosure are directed to large-scale production or collection of saccharide.

The technology disclosed in this specification pertains to methods of treating an aqueous system so as to provide a treated solution in which the concentration of phosphate is reduced, but the concentration of one or more target molecules are substantially unreduced (or substantially preserved).

In any embodiment, the target molecule is a saccharide. In any embodiment, the aqueous preparation contains a saccharide. In some embodiments, such saccharide is selected from arabinose, lyxose, ribose, xylose, ribulose, xylulose, allose, altrose, galactose, glucose, gulose, idose, mannose, talose, fructose, allulose, sorbose, tagatose and mixtures or combinations thereof. In some embodiments, such saccharide is selected from fructose, dextrose, allulose, and tagatose, or combinations thereof. In some embodiments, such saccharide is selected from allulose, and tagatose, or combinations thereof.

In some embodiments, the target molecule includes one or more starches. In some embodiments, such starches can comprise amylose, amylopectin, amylodextrin, maltodextrin, dextrin, or mixtures thereof. In some embodiments, such starches are native, modified, or combinations thereof by one or more methods, such as physical, heat, and enzymatic treatment. Such starches can be native starches or modified starches. Modified starch is defined as native starch containing amylose, amylopectin or combination of both (dent starch) which are modified using chemical, enzymatic or physical modifications. Examples of modified starch using either chemical, enzymatic or physical modifications include but are not limited to: oxidized (using an oxidizing agent to add carbonyl or carboxyl groups to the starch), phosphate (monophosphate anionic or diphosphate crosslinked), other crosslinked (adipate, epichlorohydrin), esterified (acetylated), etherified (ethylated, propylated, carboxymethyl or cationic) and combinations thereof. Such starches can be hydrolyzed by acid, enzyme or oxidant to reduce molecular weight, and can also have different base chemistry or structure from source materials (waxy, 100% amylopectin, naturally anionic phosphate). Such starches can also be dextrinized (dry roasted under acidic conditions) or pregelatinized (warm or cold water dispersible).

In further embodiments, the aqueous preparation contains one or more target molecules, such as a saccharide, in an amount of about 1 wt. % to about 90 wt. % of the aqueous preparation, about 1 wt. % to about 80 wt. % about 1 wt. % to about 70 wt. % about 1 wt. % to about 60 wt. % about 1 wt. % to about 50 wt. % about 1 wt. % to about 40 wt. % about 1 wt. % to about 30 wt. % about 1 wt. % to about 20 wt. %, or about 1 wt. % to about 10 wt. % of the aqueous preparation. In some embodiments, the aqueous preparation contains a target molecule, such as a saccharide, in an amount of about 2 wt. % to about 30 wt. % of the aqueous preparation, from about 2 wt. % to about 20 wt. %, from about 2 wt. % to about 25 wt. %, from about 2 wt. % to about 22 wt. %, or from about 2 wt. % to about 20 wt. %, from about 2 wt. % to about 10 wt. % saccharide, or at least about 2 wt. % of the aqueous preparation.

In any embodiment, the phosphate-reduced preparation retains a percentage of the target molecule present in the aqueous preparation, the percentage between about 1% to about 100% compared to the aqueous preparation, between about 10% to about 100%, between about 50% to about 100%, between about 75% to about 100%, between about 90% to about 100%, between about 50% to about 100%, between about 50% to about 90%, or between about 50% to about 75% compared to the aqueous preparation, measured as percentage by weight or percentage by volume. In any embodiment, the phosphate-reduced preparation retains between about 50% to about 100% compared to the aqueous preparation. In any embodiment, the phosphate-reduced preparation retains between about 75% to about 100% compared to the aqueous preparation. In any embodiment, the phosphate-reduced preparation retains between about 80% to about 100% compared to the aqueous preparation.

In any embodiment, the target molecule is present in the phosphate-reduced preparation in an amount measured as a percentage by weight or percentage by volume of the phosphate-reduced preparation. In any embodiment, the precipitate includes intact target molecules and degraded target molecules, for example. In any embodiment, the percentage of precipitate provides between about 1% to about 100% of the treated preparation (w/v or v/v), or about 1% to about 50%, or about 1% to about 30%, or about 1% to about 20%, or about 1% to about 10%, or about 5% to about 100%, or about 5% to about 50%, or about 5% to about 20%, or about 10% to about 100%, or about 10% to about 50%, or about 10% to about 20%, or about 10% to about 15%. In any embodiment, a treated preparation yields about 110% to about 10000% more precipitate than a preparations not subjected to treatment with polyvalent compounds (measured as w/w or w/v), 110% to about 1000%, 110% to about 500%, or about 110% to about 400%, or about 110% to about 300%, or about 110% to about 200%, or about 100 times more, about 50 times more, about 10 times more, about 5 times more, or about 2 times more.

In any embodiment, a method for making a reduced-phosphate aqueous preparation, as described in this specification includes an aqueous preparation of a chemical, biological, or biochemical nature. In any embodiment, the precipitate remains with the aqueous system. In any embodiment, the precipitate is collected and removed from the aqueous system.

In any embodiment, an aqueous system encompasses a wastewater system, where there may be stages when it is desirable to remove phosphate from the wastewater, in order to encourage the proliferation of desired microbes, to discourage the growth of undesirable microbes. In any embodiment, an aqueous system includes a chemical system where it may be desirable to remove phosphate from a solution to facilitate a chemical reaction that cannot occur in the presence of phosphate, or in the presence of certain amounts of phosphate. As another example, in some biological systems, it may be desirable to reduce phosphate levels to facilitate desired enzymatic reactions. In certain cell-free systems, it may be necessary to reduce phosphate levels to facilitate the production, concentration, or retention of other cellular components, such as saccharides, nucleic acids, or proteins. In such systems, there is a need to accomplish the reduction of phosphate levels while preserving other desired macromolecular or cellular components already resident in the system. This specification describes biological systems having reduced concentrations of phosphate that are left suitable for synthesis, manipulation, or retention of target molecules.

In any embodiment, the aqueous preparation is or exemplifies biological samples obtained from, but not limited to organism, tissues, cells, and cell-free systems. In any embodiment, the aqueous preparation is derived from isolated cells, cultured cells, or mixtures thereof. In any embodiment, a cell-free system includes cell extract-based systems, which remove components from an intact cell for external applications; and purified enzyme-based systems, which use purified components of the target molecules known to be involved in biological processes. In any embodiment, the aqueous preparation comprises a lysate of cultured cells, a single cell lysate, a mixture of cell lysates obtained from at least two cell populations, a cell-containing culture harvest, a suspension containing a cell lysate, a suspension containing lysed cells, a substantially cell-free cell culture harvest, and a partially purified protein. In any embodiment, the aqueous preparation comprises a lysate of cultured cells, a suspension containing a cell lysate, and a suspension containing lysed cells.

Exemplary cell-free systems include, but are not limited to, cell-free systems are based on Escherichia coli extracts, wheat germ extracts, rabbit reticulocyte lysates, and insect cell extracts. In any embodiment, the aqueous preparation, as described above, is obtained or derived from cells, tissues, or organisms processed by mechanical, chemical, or enzymatic lysis, or combination thereof.

In any embodiment, a cell extract for use in the present invention, known cell extracts from, E. coli, embryo of plant seed, rabbit reticulocyte, insect-derived cell and the like can be used. A cell extract can be commercially available one or prepared by a known method, and in particular, E. coli extract solutions can be prepared in accordance with the method described in Pratt, J. M. et al., Transcription and Translation, Hames, 179-209, B. D. & Higgins, S. J., eds, IRL Press, Oxford (1984).

In any embodiment, a commercially available cell extract is derived, for example, from E. coli such as E. coli S30 extract system (Promega) and RTS 500 Rapid Translation System (Roche); rabbit reticulocytes such as Rabbit Reticulocyte Lysate System (Promega), and from wheat embryo include PROTEIOS™ (TOYOBO), and mixtures thereof.

In any embodiment, aqueous preparations are obtained from prokaryotic or eukaryotic cells. In any embodiment, aqueous preparations are obtained from cells selected from, for example, a virus cell, a bacterial cell, a yeast cell, a plant cell, an animal cell, a human cell, or mixtures thereof. The cells can be obtained from prokaryotic or eukaryotic cells. In any embodiment, the aqueous preparations are obtained cell-free systems such as Escherichia coli (E. coli) extracts, wheat germ extracts, rabbit reticulocytes lysates, and insect cell extracts. In any embodiment, the aqueous preparations are obtained from E. coli cells. In any embodiment, the aqueous preparations are obtained from human cells. The most appropriate cell-free system will depend on the origin and the biochemical nature of the target molecule.

In some embodiments, partially purified cell fractions are used. A partially purified cell fraction is a cell lysate from which one or more cellular components (e.g., cell membranes) have been partially or completely removed. Such fractions may contain thermostable enzymes that keep a substantial portion of its activity after exposure to high temperatures that denature other native enzymes, or function at a relatively efficient rate after exposure to a medium to high temperature where native enzymes function at inefficient rates.

The technology disclosed in this specification pertains to a treated aqueous solution containing a reduced level of in which the concentration of phosphate is reduced, but the concentration of one or more target molecules are substantially unreduced, provided by any of the methods disclosed herein.

The technology disclosed in this specification pertains to target molecules obtained by methods of treating an aqueous system so as to provide a treated preparation or solution in which the concentration of phosphate is reduced, but the concentration of one or more target molecules are substantially unreduced (or substantially preserved or maintained). In any embodiment, the target molecule is, for example, a carbohydrate, lipid, protein, saccharide, nucleic acid, or mixtures thereof. In any embodiment, the target molecule is a saccharide, chosen from allulose, tagatose, glucose, fructose, sorbitol, ribulose, ribose, arabinose, or other saccharide. In any embodiment, the target molecule is a saccharide, chosen from allulose and tagatose.

The technology disclosed in this specification pertains to methods of treating an aqueous system so as to provide a treated preparation or solution in which the phosphate is precipitated. In any embodiment, the method provides a treated solution in which the phosphate is precipitated, but the concentration of one or more target molecules are substantially unreduced (or substantially preserved or maintained).

In any embodiment, the target molecule is a saccharide. In some embodiments, such saccharide is selected from arabinose, lyxose, ribose, xylose, ribulose, xylulose, allose, altrose, galactose, glucose, gulose, idose, mannose, talose, fructose, allulose, sorbose, tagatose and mixtures or combinations thereof. In some embodiments, such saccharide is selected from fructose, dextrose, allulose, and tagatose, or combinations thereof.

The technology disclosed in this specification pertains to methods of providing an aqueous system in which the concentration of phosphate is reduced. In any embodiment, the method provides a treated solution in which the concentration of phosphate is reduced, but the concentration of one or more target molecules are substantially unreduced (or substantially preserved).

While certain embodiments have been illustrated and described, a person with ordinary skill in the art, after reading the foregoing specification, can effect changes, substitutions of equivalents and other types of alterations to the methods, and of the present technology. Each aspect and embodiment described above can also have included or incorporated therewith such variations or aspects as disclosed regarding any or all the other aspects and embodiments.

### ASPECTS

The technology disclosed in this specification can be further understood with reference to the follow non-limiting embodiments.
1. An embodiment of reducing phosphate in an aqueous preparation comprising:
a) obtaining the aqueous preparation comprising phosphate and a target molecule;
b) combining the aqueous preparation with a first compound and a second compound, to provide a treated preparation, wherein at least one of the compounds comprises a polyvalent cation;
c) mixing the treated preparation at sufficient temperature and for sufficient time for the phosphate and the compounds to form a precipitate, to provide a reduced-phosphate preparation; and
d) optionally, removing the precipitate from the reduced-phosphate preparation;
wherein the reduced-phosphate preparation retains a portion of the target molecule.
2. The embodiment of claim 1 wherein each of the first and second compounds comprises a polyvalent cation.
3. The embodiment of any of the preceding claims, wherein the aqueous preparation maintains a pH between about 2 and about 12, between about 4 and about 10, between about 6 and about 10, between about 8 and about 10, between about 7 and about 9, between about 5.5 and about 8.5, or below about 12, or below about 10, or below about 9, or below about 8;
   preferably, wherein the aqueous preparation maintains a pH between about 5.5 and about 8.5.
4. The embodiment of any of the preceding claims, wherein the first compound comprises a polyvalent ion that is a metal ion, an alkaline earth metal ion, or a mixture thereof, and
   optionally, wherein the second compound comprises a polyvalent ion that is a metal ion, an alkaline earth metal ion, or a mixture thereof.
5. The embodiment of any of the preceding claims, wherein the polyvalent cation of the first compound is selected from calcium, magnesium, zinc, iron, titanium, and a mixture thereof; and
   optionally, the polyvalent cation of the second compound is selected from calcium, magnesium, zinc, iron, titanium, and a mixture thereof.
6. The embodiment of any of the preceding claims, wherein the first and second compounds comprise the same polyvalent cation;
   preferably, wherein the polyvalent cation is calcium.
7. The embodiment of any of the preceding claims, wherein each of the first and second compounds is selected from aluminum chloride, lanthanum chloride, calcium bromide, calcium chloride, calcium hydroxide, calcium oxide, calcium nitrate, ferric chloride, iron hydroxide, cesium nitrate, cesium chloride, cesium bromide, magnesium chloride, magnesium hydroxide, magnesium oxide, magnesium bromide, hydrogen chloride, sulfuric acid, ammonium hydroxide sodium hydroxide, potassium hydroxide, zinc chloride, zinc bromide, and mixtures thereof,
   preferably, wherein the first and second compounds are selected from calcium chloride, calcium hydroxide, and calcium oxide.
8. The embodiment of any of the preceding claims, wherein the treated preparation comprises the first compound at between about 0.001 wt. % to about 10.0 wt. % of the treated preparation, or about 0.01 wt. % to about 10.0 wt. %, about 0.01 wt. % to about 1.0 wt. %, about 0.50 wt. % to about 1.0 wt. %, or about 0.50 wt. % to about 0.9 wt. % of the treated preparation;
   preferably, wherein the treated preparation comprises the first compound at between about 0.01 wt. % to about 10.0 wt. % of the treated preparation.
9. The embodiment of any of the preceding claims, wherein the treated preparation comprises the second compound at between about 0.001 wt. % to about 10.0 wt. % of the treated preparation, or about 0.01 wt. % to about 10.0 wt. %, about 0.1 wt. % to about 5.0 wt. %, about 0.5 wt. % to about 2.0 wt. %, or about 0.50 wt. % to about 1.0 wt. % of the treated preparation;
   preferably, wherein the treated preparation comprises the second compound at between about 0.01 wt. % to about 10.0 wt. % of the treated preparation.
10. The embodiment of any of the preceding claims, wherein the treated preparation comprises the first and second compounds in a ratio of between about 0.5:1 to about 7:1, between about 0.5:1 to about 5:1, between about 0.5:1 to about 5:1, between about 0.5:1 to about 3:1, between about 0.5:1 to about 1:1, between about 1:0 to about 2:0, or about 1:1, or about 1:1.2, or about 1:1.4, or about 1:1.6, or about 1:1.8, or about 1:1.2.
11. The embodiment of any of the preceding claims, wherein the aqueous preparation comprises soluble phosphate in an amount between about 1 and about 1000 mM, between about 1 and about 500 mM, between about 1 and about 250 mM, between about 1 and about 100 mM, between about 10 and about 1000 mM, between about 10 and about 500 mM, between about 10 and about 250 mM, between about 10 and about 100 mM, or between about 10 and about 50 mM.
12. The embodiment of any of the preceding claims, wherein the treated preparation has a pH between about 2 and about 12, between about 4 and about 10, between about 6 and about 10, between about 8 and about 10, between about 9 and about 10, or below about 12, or below about 10, or below about 9, or below about 8;
   preferably, wherein the treated preparation has a pH between about between about 6 and about 10.
13. The embodiment of any of the preceding claims,
   wherein the mixing step is performed at a temperature between about 10° C. and about 200° C., between about 10° C. and about 150° C., between about 10° C. and about 120° C., between about 20° C. and about 100° C., between about 40° C. and about 80° C., between about 60° C. and about 80° C., between about 60° C. and about 70° C., or between about 10° C. and about 100° C., or about 60° C., or about 65° C., or about 70° C., or about 75° C.; and
   optionally, for a time between about 1 minute to about 60 minutes, between 5 minutes to about 60 minutes, between about 10 minutes to about 60 minutes, between about 15 minutes to about 60 minutes, between about 10 minutes to about 45 minutes, between about 10 minutes to about 30 minutes, or about 5 minutes, about 10 minutes, about 15 minutes, about 20 minutes, about 30 minutes, or about 60 minutes.
14. The embodiment of any of the preceding claims, wherein after sufficient temperature and for sufficient time for the phosphate and the compounds to combine as a precipitate, the precipitate comprises from about 1 wt. % to about 100 wt. % of the phosphate in the treated preparation, or from about 1 wt. % to about 90 wt. %, or from about 1 wt. % to about 80 wt. %, or from about 1 wt. % to about 70 wt. %, or from about 1 wt. % to about 60 wt. %, or from about 10 wt. % to about 100 wt. %, from about 10 wt. % to about 90 wt. %, or from about 10 wt. % to about 80 wt. %, or from about 10 wt. % to about 70 wt. %, or from about 10 wt. % to about 50 wt. %, or from about 50 wt. % to about 100 wt. %, or from about 50 wt. % to about 90 wt. %, or from about 50 wt. % to about 80 wt. % of the phosphate in the treated preparation.
15. The embodiment of any of the preceding claims, wherein the precipitate is removed by a process selected from the following: filtration, centrifugation, and precipitation.
16. The embodiment of any of the preceding claims, the reduced-phosphate preparation retains a percentage of the phosphate from the aqueous preparation at between about 1% to about 20%, between about 1% to about 10%, between about 1% to about 5%, or between about 5% to about 10%;
   preferably, wherein the reduced-phosphate preparation retains a percentage of the phosphate from the aqueous preparation at between about 1% to about 20%.
17. The embodiment of any of the preceding claims, wherein the target molecule is a saccharide.
18. The embodiment of claim 17, wherein the saccharide is selected from arabinose, lyxose, ribose, xylose, ribulose, xylulose, allose, altrose, galactose, glucose, gulose, idose, mannose talose, fructose, allulose, sorbose, tagatose and mixtures and combinations thereof;
   preferably, wherein the saccharide is selected from allulose and tagatose.
19. The embodiment of any of claims 17-18, wherein the aqueous preparation contains at least about 2 wt. % of saccharide, or from about 2 wt. % to about 30 wt. %, from about 2 wt. % to about 25 wt. %, from about 2 wt. % to about 22 wt. %, or from about 2 wt. % to about 10 wt. % of saccharide.
20. The embodiment of any of claims 1-19, wherein the reduced-phosphate preparation retains a percentage of the target molecule present in the aqueous preparation, the percentage between about 1 wt. % to about 100 wt. %, between about 10 wt. % to about 100 wt. %, between about 50 wt. % to about 100 wt. %, between about 75 wt. % to about 100 wt. %, between about 90 wt. % to about 100 wt. %, between about 50 wt. % to about 100 wt. %, between about 50 wt. % to about 90 wt. %, or between about 50 wt. % to about 75 wt. %; and
   preferably, wherein reduced-phosphate preparation retains between about 80 wt. % to about 100 wt. % of the target molecule present in the aqueous preparation.
21. The embodiment of any of the preceding claims, wherein the aqueous preparation comprises a starch.
22. The embodiment of any of the preceding claims, wherein, optionally, the starch comprises amylose, amylopectin, amylodextrin, maltodextrin, or mixtures thereof.
23. The embodiment of any of the preceding claims, wherein the aqueous preparation is derived from cultured cells, and is selected from the group consisting of a lysate of cultured cells, a single cell lysate, a mixture of cell lysates obtained from at least two cell populations, a cell-containing culture harvest, a suspension containing a cell lysate, a suspension containing lysed cells, a substantially cell-free cell culture harvest, and a partially purified protein.
24. The embodiment of claim 23, wherein the cell is selected from a bacterial cell, a yeast cell, a plant cell, an animal cell, a human cell, and mixtures thereof.
25. The embodiment of any of the preceding claims, wherein the aqueous preparation is derived from cells processed by mechanically, chemically, or enzymatically lysing the cells.
26. The embodiment as described in any of the preceding claims, wherein the embodiment makes reduced-phosphate aqueous preparation as described in any preceding claim.
27. The embodiment of any of the preceding claims, wherein each of the first and second compounds provides a content of at least about 0.5 wt. % to about 1.5 wt. % of the aqueous system, and together provides a reduction of at least about 80% in the phosphate concentration in aqueous system.
28. A reduced-phosphate aqueous preparation provided by the embodiment of any one of the preceding claims.
29. Use of the reduced-phosphate aqueous preparation as recited in any one of claims 17-28 to provide a saccharide.
30. A saccharide prepared by the embodiment of any one of claims 17-28.
31. Use of the saccharide of claim 31.

The technology disclosed in this specification can be further understood with reference to the following examples, which are not intended to be limiting in any way.

### EXAMPLES

### Example 1: Preparation of Cell Lysate Suspension

An aqueous suspension approximating an aqueous biological suspension provided by a cell-free lysate was prepared from the components listed in Table 1. Solid saccharides (allulose, fructose, and glucose) were dissolved in deionized water, at room temperature. Salts were added to the mixture: MgCl₂, MnCl₂, CoCl₂, NaCl, and NaH₂PO₄. The aqueous suspension contained the listed components in the amounts specified in Table 1.

A volume of cell lysate was added to the solution, the cell lysate obtained from Escherichia co/i. (E. coli) microbes were heat-treated to kill the bacteria, and then homogenized; the resulting lysate was kept frozen at −80° C. until thawed and used in preparing the cell lysate suspension. The cell lysate contained proteins, nucleic acids, polysaccharides, and other soluble or insoluble cellular structures soluble or insoluble.

The volume of the cell lysate suspension was adjusted by the addition of deionized water, providing an exemplary cell lysate suspension. If needed, the pH of the cell lysate suspension was adjusted to provide pH 6.5 to the solution. Table 1 shows the composition of an exemplary cell-free aqueous suspension.

TABLE 1
Composition of Cell Lysate Suspensions

| Component | Amount (g/L) |
|---|---|
| Maltodextrin | 79.1 |
| Allulose | 123 |
| Fructose | 14 |
| Glucose | 6 |
| MgCl₂ | 0.19 |
| MnCl₂ | 0.25 |
| CoCl₂ | 0.065 |
| NaCl | 0.185 |
| NaH₂PO₄ | 2.52 |
| Cell lysate (E. coli) | 350 mL cell lysate (containing |
| | about 60 g dry cell matter) |
| Water | Bring total volume to 1 liter |

### Example 2: Reduction of Phosphate from Cell Lysate Suspension

Solutions of 40% (w/v) CaCl₂) and 20% (w/v) Ca(OH)₂ were prepared. Samples of cell lysate suspension were obtained. The cell lysate suspension samples were preheated to 65° C. To each sample, amounts of polyvalent compounds CaCl₂) and Ca(OH)₂ were added, as shown in Table 2. The polyvalent compounds were added so as to maintain the pH of the cell lysate suspension at a pH in a range between about 4 and about 12 during the addition and admixture of the polyvalent calcium polyvalent compounds into the cell lysate suspension.

TABLE 2
Polyvalent Compounds Added to Cell Lysate Suspensions

| | | g CaCl₂ | g Ca(OH)₂ | CaCl₂ | Ca(OH)₂ |
|---|---|---|---|---|---|
| No. | Description | (40 wt. %) | (20 wt. %) | (% (w/w)) | (% (w/w)) |
| 0a | Unheated | 0 | 0 | 0 | 0 |
| 0b | Heated to 60° C. | 0 | 0 | 0 | 0 |
| 1 | | 0.625 | 1.5 | 0.13% | 0.15% |
| 2 | | 0 | 0 | 0 | 0 |
| 3 | | 1.66 | 4.5 | 0.33% | 0.45% |
| 4 | | 0 | 4.5 | 0 | 0.45% |
| 5 | | 0.83 | 2.25 | 0.17% | 0.23% |
| 6 | | 0.83 | 2.25 | 0.17% | 0.23% |
| 7 | | 1.66 | 0 | 0.33% | 0 |
| 8 | | 0.75 | 2 | 0.15% | 0.20% |
| 9 | | 0.83 | 4.5 | 0.17% | 0.45% |
| 10 | | 0.83 | 0 | 0.17% | 0 |
| 11 | | 0 | 2.25 | 0 | 0.23% |
| 12 | | 0.83 | 1.1 | 0.17% | 0.11% |
| 13 | | 0 | 1.1 | | 0.11% |
| 14 | | 1.66 | 2.25 | 0.33% | 0.23% |
| 15 | | 0 | 0 | 0 | 0 |

The admixtures were agitated during the addition of the polyvalent compounds and for an additional 15 minutes afterward, to encourage the formation of complexes between the polyvalent compounds and the soluble phosphates and not only in the suspension.

Portions of the solutions were subjected to centrifugation (2000×g, 10 min) to collect precipitates or complexes. The resulting supernatants of the centrifuged solutions were collected by filtration through diatomaceous earth (such as Celite 545 or Kenite 5200) supported on Whatman paper with 8 um or similar sized pores to separate the precipitates from the supernatants. The filtered supernatants were analyzed to determine phosphate levels, as determined by malachite green phosphate assay (Science Cell™ Research Laboratories), a colorimetric method for the measurement of inorganic, soluble phosphate concentrations. The samples were measured in duplicate.

The filtered supernatant solutions were stored at 4° C. Without being bound to theory, it is believed that the precipitates collected by centrifugation and filtration contained chelated inorganic phosphate and cellular debris from the lysed E. coli that was the source of the cell lysate component.

The pH of each sample was typically measured with Accumet Basic model AB15 or similar instrument and the conductivity was typically measured with Traceable model 89094-958 or similar portable or stationary instruments. After addition of the admixture of the calcium polyvalent compounds, the pH and the conductivity of the treated solutions were measured, as shown in Table 3.

TABLE 3
Physical Properties of Treated Cell Lysate Suspensions

| | CaCl₂ | Ca(OH)₂ | Phosphate | | Conductivity |
|---|---|---|---|---|---|
| No. | (% (w/w)) | (% (w/w)) | (mM) | pH | (mS/cm) |
| 0a | 0 | 0 | 27.8 ± 0.9 | 7.0 | 4.5 |
| 0b | 0 | 0 | 26.3 ± 3.4 | 7.0 | 4.5 |
| 2 | 0 | 0 | 25.6 ± 2.4 | 6.8 | 7.1 |
| 15 | 0 | 0 | 25.5 ± 1.1 | 6.8 | 7.02 |
| 10 | 0.83% | 0 | 15.3 ± 1.8 | 4.8 | 9.0 |
| 7 | 1.66% | 0 | 15.3 ± 1.1 | 4.4 | 11.4 |
| 13 | 0 | 0.55% | 13.0 ± 1.4 | 9.6 | 7.0 |
| 11 | 0 | 1.13% | 2.9 ± 0.4 | 10.1 | 6.7 |
| 4 | 0 | 2.25% | 3.0 ± 0.4 | 10.5 | 7.9 |
| 1 | 0.63% | 0.75% | 3.0 ± 0.5 | 6.9 | 4.7 |
| 8 | 0.75% | 1.00% | 1.8 ± 1.8 | 9.8 | 8.9 |
| 12 | 0.83% | 0.55% | 3.4 ± 0.8 | 8.0 | 8.2 |
| 5 | 0.83% | 1.13% | 1.4 ± 0.6 | 9.9 | 8.9 |
| 6 | 0.83% | 1.13% | 1.3 ± 0.5 | 9.9 | 9.1 |
| 9 | 0.83% | 2.25% | 3.1 ± 2.1 | 10.3 | 10.2 |
| 14 | 1.66% | 1.13% | 1.2 ± 0.5 | 9.6 | 11.5 |
| 3 | 1.66% | 2.25% | 2.3 ± 0.2 | 10.3 | 12.3 |

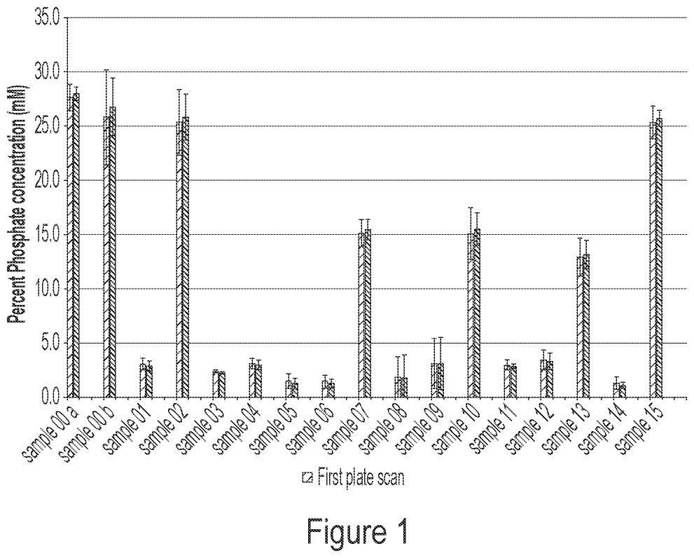

As shown in FIG. 1 and Table 3, the cell lysate solutions contained phosphate prior to treatment with polyvalent salts (samples 0a, 0b, 2, and 15). Treatment with CaCl₂) alone (samples 7 and 10) provided for reduced phosphate levels; treatment with Ca(OH)₂, alone (samples 4, 11, 13) also reduced phosphate levels. Treatment with both salts provided further reduced the phosphate levels, compared to treatment with a single salt (samples 1, 3, 5,-6, 8-9, 12, and 14).

The addition of minimal amounts of CaCl₂) alone (sample 10) or minimal amounts of Ca(OH)₂ alone (sample 13) resulted in treated aqueous suspensions having concentrations of phosphate of about 11-18 mM phosphate, which was smaller than untreated control samples (samples 0a, 0b, 2, 15) which had phosphate concentrations of about 25-28 mM. The addition of greater amounts of CaCl₂) alone (sample 7) or minimal amounts of Ca(OH)₂ alone (samples 11, 4) provided reduced phosphate concentrations of phosphate, about 15 mM and about 3 mM, respectively.

Where a single polyvalent compound was used, increasing concentrations of the polyvalent compounds provided lower levels of phosphate. The addition of different concentrations of combined CaCl₂) and Ca(OH)₂ (samples 1, 3, 5, 6, 8-9, 12, 14) resulted in decreased concentrations of phosphate of approximately 1-4 mM phosphate.

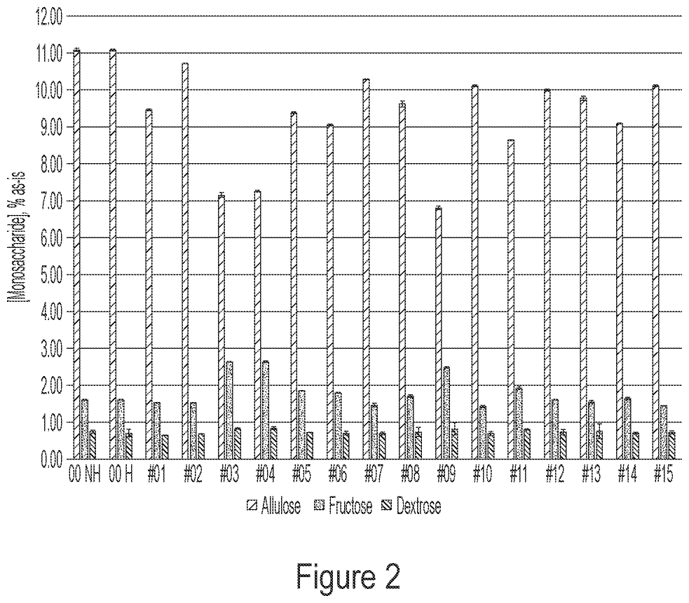

As shown in FIG. 2 and Table 3, the greatest reduction of phosphate was achieved with combined treatment with CaCl₂) and Ca(OH)₂, compared to treatment with only one of the polyvalent salts. But when the polyvalent salts were combined, there did not appear to be a linear relationship between the amounts the combined polyvalent compounds and the removal of the phosphates. As shown in FIG. 2 and Table 3, the supernatants were analyzed to determine the carbohydrate levels in the samples. The samples were subjected to HLPC to quantify the amounts of different saccharides (allulose, fructose, dextrose) in each sample.

Where phosphate reduction was accomplished with CaCl₂) alone, the treatment provided for allulose levels comparable to controls (samples 7 and 10 versus samples 0a, 0b, 2, 15). Where phosphate reduction was accomplished with Ca(OH)₂ alone, the increasing amounts of polyvalent compound provided for decreasing amounts of allulose compared to controls (samples 4, 11, 13 versus samples 0a, 0b, 2, 15).

Where phosphate reduction was accomplished with both polyvalent compounds, increasing amounts of combined polyvalent compounds provided a non-linear relationship in the amounts of allulose compared to controls (samples 1, 5-6, 3, 12, 8-9, 14) versus samples 0a, 0b, 2, 15). Maximum retention of allulose was observed in samples 8 and 12.

### Example 3: Retention of Saccharides with Phosphate Treatment

Along with the reduction of phosphate levels, the samples of Example 2 were also screened for the retention of various saccharides, as shown in FIG. 2 and Table 4.

Certain combinations of the polyvalent compounds provided for reduced loss of allulose during the method of treatment. In the absence of polyvalent compounds, allulose levels were present at about 10-11 wt. %, compared to the weight of the combined dry components. The addition of polyvalent compounds sufficient to reduce the phosphate levels below about 10 mM also resulted in a reduction in the amount of allulose in the treated suspension, to levels generally less than about 10 wt. %.

Superior retention of allulose (final allulose concentrations above about 9 wt. %), concurrent with the removal of phosphate, occurred at preparations treated with combined polyvalent compound treatments of about 0.63 to about 0.83 wt. % CaCl₂) and about 0.55 to about 1.13 wt. % Ca(OH)₂, compared to the dry weight of the cell lysate components (samples 1, 5, 6, 8, and 12). These conditions provided pHs between about 6.9 to about 9.9. Optimal phosphate reduction (below 10 mM) and optimal allulose retention (about 10 wt. %) were observed when about 0.83 wt. % CaCl₂) and about 0.55 wt. % Ca(OH)₂ were used as polyvalent compounds (sample 12), which provided a pH about 8.0.

As shown in Table 3, the treatments applied to samples 5, 6, 8, and 12 provided conductivities between about 4.7 and about 9.1 mS/cm.

TABLE 4
Saccharide Retention in Treated Cell Lysate Suspensions

| | CaCl₂ | | | | | | |
|---|---|---|---|---|---|---|---|
| | (% | Ca(OH)₂ | Phosphate | | Allulose | Fructose | Dextrose |
| No. | (w/w)) | (% (w/w) | (mM) | pH | wt. % | wt. % | wt. % |
| 0a | 0 | 0 | 27.8 ± 0.9 | 7.0 | 11.10 | 1.61 | 0.75 |
| 0b | 0 | 0 | 26.3 ± 3.4 | 7.0 | 11.09 | 1.61 | 0.70 |
| 2 | 0 | 0 | 25.6 ± 2.4 | 6.8 | 10.72 | 1.52 | 0.69 |
| 15 | 0 | 0 | 25.5 ± 1.1 | 6.8 | 10.12 | 1.44 | 0.72 |
| 10 | 0.83% | 0 | 15.3 ± 1.8 | 4.8 | 10.11 | 1.42 | 0.70 |
| 7 | 1.66% | 0 | 15.3 ± 1.1 | 4.4 | 10.30 | 1.46 | 0.70 |
| 13 | 0 | 0.55% | 13.0 ± 1.4 | 9.6 | 9.78 | 1.55 | 0.76 |
| 11 | 0 | 1.13% | 2.9 ± 0.4 | 10.1 | 8.64 | 1.93 | 0.80 |
| 4 | 0 | 2.25% | 3.0 ± 0.4 | 10.5 | 7.26 | 2.64 | 0.84 |
| 1 | 0.63% | 0.75% | 3.0 ± 0.5 | 6.9 | 9.46 | 1.53 | 0.64 |
| 8 | 0.75% | 1.00% | 1.8 ± 1.8 | 9.8 | 9.63 | 1.70 | 0.74 |
| 12 | 0.83% | 0.55% | 3.4 ± 0.8 | 8.0 | 10.00 | 1.49 | 0.74 |
| 5 | 0.83% | 1.13% | 1.4 ± 0.6 | 9.9 | 9.38 | 1.84 | 0.73 |
| 6 | 0.83% | 1.13% | 1.3 ± 0.5 | 9.9 | 9.05 | 1.80 | 0.71 |
| 9 | 0.83% | 2.25% | 3.1 ± 2.1 | 10.3 | 6.80 | 2.48 | 0.82 |
| 14 | 1.66% | 1.13% | 1.2 ± 0.5 | 9.6 | 9.10 | 1.64 | 0.70 |
| 3 | 1.66% | 2.25% | 2.3 ± 0.2 | 10.3 | 7.15 | 2.64 | 0.84 |

1. A method of reducing phosphate in an aqueous preparation comprising:
a) obtaining the aqueous preparation comprising phosphate and a target molecule;
b) combining the aqueous preparation with a first compound and a second compound, to provide a treated preparation, wherein both compounds comprises a polyvalent cation;
c) mixing the treated preparation at sufficient temperature and for sufficient time for the phosphate and the compounds to form a precipitate, to provide a reduced-phosphate preparation; and
d) optionally, removing the precipitate from the reduced-phosphate preparation;
wherein the aqueous preparation maintains a pH between about 4 and about 10 and the treated preparation has a pH between about 4 and about 10;
wherein the reduced-phosphate preparation retains a portion of the target molecule;
wherein the target molecule is a saccharide selected from the group consisting of arabinose, lyxose, ribose, xylose, ribulose, xylulose, allose, altrose, galactose, glucose, gulose, idose, mannose talose, fructose, allulose, sorbose, tagatose and mixtures and combinations thereof.

2. (canceled)

3. (canceled)

4. (canceled)

5. The method claim 1, wherein the polyvalent cation of the first compound is selected from the group consisting of calcium, magnesium, zinc, iron, titanium, and a mixture thereof; and
optionally, the polyvalent cation of the second compound is selected from the group consisting of calcium, magnesium, zinc, iron, titanium, and a mixture thereof.

6. The method of claim 1, wherein the first and second compounds comprise the same polyvalent cation;
optionally, wherein the polyvalent cation is calcium.

7. The method of claim 1, wherein each of the first and second compounds is selected from group consisting of aluminum chloride, lanthanum chloride, calcium bromide, calcium chloride, calcium hydroxide, calcium oxide, calcium nitrate, ferric chloride, iron hydroxide, cesium nitrate, cesium chloride, cesium bromide, magnesium chloride, magnesium hydroxide, magnesium oxide, magnesium bromide, hydrogen chloride, sulfuric acid, ammonium hydroxide sodium hydroxide, potassium hydroxide, zinc chloride, zinc bromide, and mixtures thereof.

8. The method of claim 1, wherein the treated preparation comprises the first compound at between about 0.001 wt. % to about 10.0 wt. % of the treated preparation.

9. The method of claim 1, wherein the treated preparation comprises the second compound at between about 0.001 wt. % to about 10.0 wt. % of the treated preparation.

10. The method of claim 1, wherein the treated preparation comprises the first and second compounds in a ratio of between about 0.5:1 to about 7:1.

11. The method of claim 1, wherein the aqueous preparation comprises soluble phosphate in an amount between about 1 and about 1000 mM.

12. (canceled)

13. (canceled)

14. The method of claim 1, wherein after sufficient temperature and for sufficient time for the phosphate and the compounds to combine as a precipitate, the precipitate comprises from about 1 wt. % to about 100 wt. % of the phosphate in the treated preparation.

15. The method of claim 1, wherein the precipitate is removed by a process selected from the following: filtration, centrifugation, and precipitation.

16. The method of claim 1, the reduced-phosphate preparation retains a percentage of the phosphate from the aqueous preparation at between about 1% to about 20%.

17. (canceled)

18. (canceled)

19. The method of claim 1, wherein the aqueous preparation contains at least about 2 wt. % of saccharide.

20. The method of claim 1, wherein the reduced-phosphate preparation retains a percentage of the target molecule present in the aqueous preparation.

21. (canceled)

22. (canceled)

23. The method of claim 1, wherein the aqueous preparation is derived from cultured cells, and is selected from the group consisting of a lysate of cultured cells, a single cell lysate, a mixture of cell lysates obtained from at least two cell populations, a cell-containing culture harvest, a suspension containing a cell lysate, a suspension containing lysed cells, a substantially cell-free cell culture harvest, and a partially purified protein.

24. The method of claim 1, wherein the aqueous preparation is derived from cultured cells and the cell is selected from the group consisting of a bacterial cell, a yeast cell, a plant cell, an animal cell, a human cell, and mixtures thereof.

25. The method of claim 1, wherein the aqueous preparation is derived from cells processed by mechanically, chemically, or enzymatically lysing the cells.

26. (canceled)

27. The method of claim 1, wherein each of the first and second compounds provides a content of at least about 0.5 wt. % to about 1.5 wt. % of the aqueous system.

28. (canceled)

29. (canceled)

30. (canceled)

31. (canceled)

32. The method of claim 1 wherein the method provides a reduction of at least about 80% in the phosphate concentration in aqueous preparation.
